(19) United States
(12) Patent Application Publication
(10) Pub. No.: US 2005/0085416 A1
(43) Pub. Date: Apr. 21, 2005

(54) Biologically active complex

(76) Inventors: Catharina Svanborg, Lund (SE); Malin Wilhelmina Svensson, Lund (SE)

Correspondence Address: NIXON & VANDERHYE, PC, 1100 N GLEBE ROAD, 8TH FLOOR, ARLINGTON, VA, 22201-4714, US

(21) Appl. No.: 10/506,903

(22) PCT Filed: Mar. 7, 2003
(86) PCT No.: PCT/IB03/01293

(30) Foreign Application Priority Data
Mar. 7, 2002 (GB) 0205347.8

(51) Int. Cl. A61K 38/38; A61K 31/202

(52) U.S. Cl. 514/12; 514/560

(57) ABSTRACT

A biologically active complex comprising alpha-lactalbumin or a variant of alpha-lactalbumin which is in the apo folding state, or a fragment of either of any of these, and a cofactor which stabilizes the complex in a biologically active form, provided that any fragment of alpha-lactalbumin or a variant thereof comprises a region corresponding to the region of α-lactalbumin which forms the interface between the alpha and beta domains, and further provided that when the complex comprises native alpha-lactalbumin, the cofactor is other than C18:1:9 cis fatty acid. These complexes have therapeutic applications for example in the treatment of cancer and as antibacterial agents.

The present invention relates to biologically active complexes, in particular complexes derived from alpha-lactalbumin α-lactalbumin), to pharmaceutical compositions containing these as well as to their use in therapy, in particular as anti-cancer or antibacterial agents.

Biologically active complexes obtained from milk and particularly human milk, together with their use as antibacterial agents is described for example in EP-0776214.

HAMLET (formerly known as MAL) is a molecular complex that induces in vitro apoptosis selectively in tumour cells, but not in healthy differentiated cells. The apoptotic activity of this variant fold was discovered by serendipity, in a fraction of human milk casein obtained by precipitation at low pH, and was purified by ion exchange chromatography, eluting as a single peak after 1M NaCl. The elute was shown by spectroscopy to contain partially unfolded α-lactalbumin in an apo-like conformation (M. Svensson, et al, (1999) J Biol Chem, 274, 6388-96), with native-like secondary structure, but lacking specific tertiary packing of the side chains. The link between apoptosis induction and the folding change was proven by deliberate conversion of native α-lactalbumin to the apoptosis inducing form (M. Svensson, et al., (2000) Proc Natl Acad Sci USA, 97, 4221-6). HAMLET was shown to bind to the surface of tumour cells, to translocate into the cytoplasm and to accumulate in cell nuclei, where it causes DNA fragmentation (M. Svensson, et al., (2000) Proc Natl Acad Sci USA, 97, 4221-6).

It has also been found that other reagents and specifically lipid such as oleic acid, are useful in the conversion of human α-lactalbumin to HAMLET (human α-lactalbumin made lethal to tumour cells). In particular, it has been reported previously that oleic acid (C18:1:9cis) is required for HAMLET production (M. Svensson, et al., (2000) Proc Natl Acad Sci USA, 97, 4221-6).

Five of the seven oxygens that ligate the Ca²⁺ are contributed by side chain carboxylates of Asp residues at positions 82, 87 and 88 and by carbonyl oxygens of Lys 79 and Asp 84, and two water molecules supply the remaining ligands (Acharya et al., 1991). The bound Ca²⁺ brings the α-helical region and the β-sheet in close proximity, and two disulfide bonds flanking the Ca²⁺ binding site, make this part of the molecule fairly inflexible.

α-Lactalbumin is the dominant protein in human milk, where it is present at a concentration of 2 mg/ml (140 μM). The mature protein consists of 123 amino acid residues (14.2 kDa). Its three dimensional structure has been determined to 1.7 A resolution and it is a globular protein with four α-helices (residues 1-34, 86-123) and a triple stranded anti-parallel β-sheet (residues 38-82), linked by four disulphide bonds (61-77; 73-91; 28-111 and 6-120) (K. R. Acharya, et al., (1991) J Mol Biol, 221, 571-81). Binding of Ca²⁺ to a single very high affinity Ca²⁺ binding site is required for the protein to maintain a native conformation. Five of the seven oxygens that ligate the Ca are contributed by side chain carboxylates of Asp residues at positions 82, 87 and 88 and by carbonyl oxygens of Lys 79 and Asp 84, and two water molecules supply the remaining ligands. The bound Ca²⁺ brings the α-helical region and the β-sheet in close proximity, and two disulfide bonds flanking the Ca²⁺ binding site, make this part of the molecule fairly inflexible.

The protein adopts the so called apo state found in HAMLET when exposed to low pH, or in the presence of chelators, that release the strongly bound Ca²⁺ ion (D. A. Dolgikh, et al., (1981) FEBS Lett, 136, 311-5; K. Kuwajima, (1996) Faseb J, 10, 102-09).

The applicants have found that the conversion of α-lactalbumin to HAMLET with apoptotic activity, requires both a conformational or folding change and the presence of a lipid cofactor and this may preferably be achieved using a variant of alpha-lactalbumin. The conformational or folding change is conveniently effected by removal of calcium ions, or by using a variant without calcium ions. However, once the change has been effected, the presence of calcium or a functional calcium binding site does not result in any loss of activity.

Furthermore, they have found that the optimal cofactors for the conversion of alpha-lactalbumin to HAMLET are C18:1 fatty acids with a double bond in the cis conformation at position 9 or 11. Saturated C18 fatty acid or unsaturated fatty acids in the trans conformation, or fatty acids with shorter carbon chains did not form HAMLET, suggesting that highly specific inter-molecular interactions are required for lipids to act as folding partners in this system.

According to the present invention there is provided a biologically active complex comprising alpha-lactalbumin or a variant of alpha-lactalbumin which is in the apo folding state, or a fragment of either of any of these, and a cofactor which stabilises the complex in a biologically active form, provided that any fragment of alpha-lactalbumin or a variant thereof comprises a region corresponding to the region of alpha-lactalbumin which forms the interface between the alpha and beta domains, and further provided that when the complex comprises native alpha-lactalbumin, the cofactor is other than C18:1:9 cis fatty acid.

In particular the cofactors are selected from a cis C18:1:9 or C18:1:11 fatty acid or a different fatty acid with a similar configuration.

The complex is suitably prepared by forming the apo conformation of the protein, using conventional or molecular biological methods, and in particular by removing calcium ions from alpha-lactalbumin or variants, or by using variants from which calcium ions have been released, or which do not have a functional calcium binding site.

However, the applicants have found that, once formed, the presence of a functional calcium binding site, and/or the presence of calcium, does not affect stability or the biological activity of the complex. Biologically active complexes have been found to retain affinity for calcium, without loss of activity.

Therefore complex of the invention may further comprise calcium ions but the elimination of calcium from the complex is not essential, but provides a convenient means for the preparation of the complex.

Thus, in a particular embodiment, there is provided a biologically active complex which is obtainable by combining
(i) a cis C18:1:9 or C18:1:11 fatty acid or a different fatty acid with a similar configuration; and
(ii) alpha-lactalbumin from which calcium ions have been removed, or a variant of alpha-lactalbumin from which calcium ions have been released or which does not have a functional calcium binding site.; or a fragment of either of any of these, provided that any fragment comprises a region corresponding to the region of α-lactalbumin which forms the interface between the alpha and beta domains, and further provided that when (ii) is alpha-lactalbumin, (i) is other than C18:1:9 cis fatty acid.

In particular, the complex will comprise elements (i) and (ii).

The expression “biological active” of “biological activity” as used herein means that the complex has similar biological activity to that reported for HAMLET. In other words, it will be effective in producing apopotosis in cancer cells and/or have antibacterial properties.

As used herein the expression “variant” refers to polypeptides or proteins which are homologous to the basic protein, which is suitably human or bovine α-lactalbumin, but which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide suitably variants will be at least 60% identical, preferably at least 70%, even more preferably 80% or 85% and, especially preferred are 90%, 95% or 98% or more identity.

When comparing amino acid sequences for the purposes of determining the degree of identity, programs such as BESTFIT and GAP (both from Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of sequences, the comparison is made by alignment of the sequences along their whole length.

The term “fragment thereof” refers to any portion of the given amino acid sequence which will form a complex with the similar activity to complexes including the complete α-lactalbumin amino acid sequence. Fragment may comprise more than one portion from within the full-length protein, joined together. Portions will suitably comprise at least 5 and preferably at least 10 consecutive amino acids from the basic sequence.

Suitable fragments will be deletion mutants suitably comprise at least 20 amino acids, and more preferably at least 100 amino acids in length. They include small regions from the protein or combinations of these.

In a particularly preferred embodiment, the variant used in the method of the invention is one in which the calcium binding site has been modified so that the affinity for calcium is reduced, or it is no longer functional. It has been found that in bovine α-lactalbumin, the calcium binding site is coordinated by the residues K79, D82, D84, D87 and D88. Thus modification of this site, for example by removing one of more of the acidic residues, can reduce the affinity of the site for calcium, or eliminate the function completely and mutants of this type are a preferred aspect of the invention.

The Ca²⁺-binding site of bovine α-lactalbumin consists of a 3₁₀ helix and an α-helix with a short turn region separating the two helices (Acharya K. R., et al., (1991) J Mol Biol 221, 571-581). It is flanked by two disulfide bridges making this part of the molecule fairly inflexible. Five of the seven oxygen groups that co-ordinate the Ca²⁺ are contributed by the side chain carboxylates of Asp82, 87 and 88 or carbonyl oxygen's of Lys79 and Asp84. Two water molecules supply the remaining two oxygen's (Acharya K. R., et al., (1991) J Mol Biol 221, 571-581).

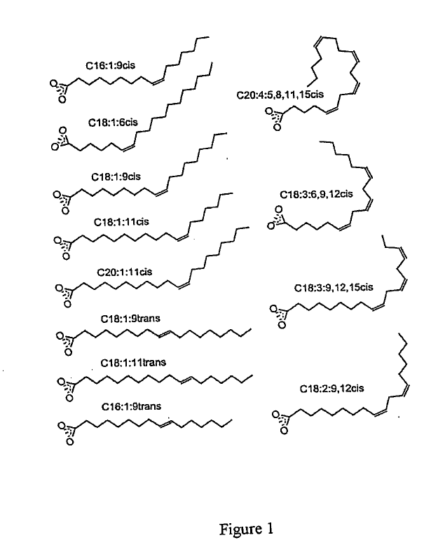

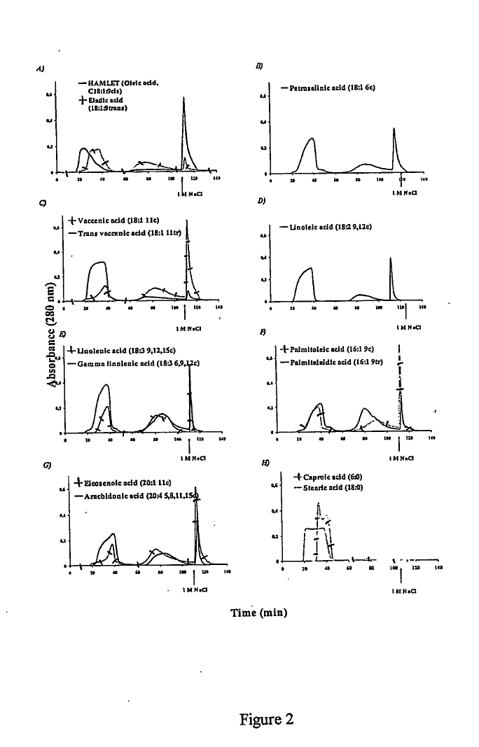

Site directed mutagenesis of the aspartic acid at position 87 to alanine (D87A) has previously been shown to inactivate the strong calcium-binding site (Anderson P. J., et al., (1997) Biochemistry 36, 11648-11654) (FIG. 1a) and the mutant proteins adopted to the apo-conformation.

Therefore in a particular embodiment, the aspartic acid residue at amino acid position 87 within the protein sequence is mutated to a non-acidic residue, and in particular a non-polar or uncharged polar side chain.

Non-polar side chains include alanine, glycine, valine, leucine, isoleucine, proline, phenylalanine, methionine, tryptophan or cysteine. A particularly preferred example is alanine.

Uncharged polar side chains include asparagine, glutamine, serine, threonine or tyrosine.

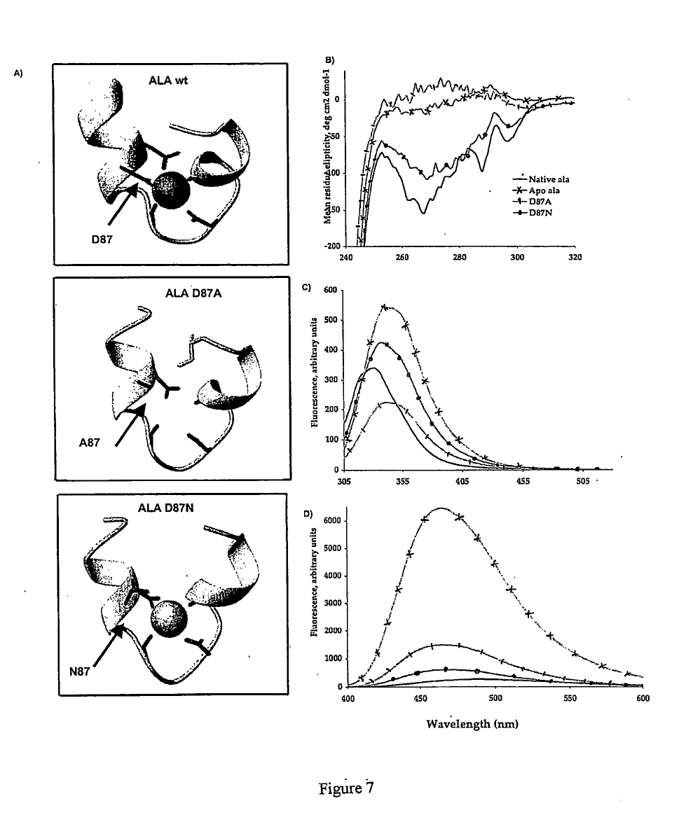

In order to minimize the structural distortion in the mutant protein, D87 has also been replaced by an asparagine (N) (Permyakov S. E., et al., (2001) Proteins Eng 14, 785-789), which lacks the non-compensated negative charge of a carboxylate group, but has the same side chain volume and geometry (FIG. 7a). The mutant protein (D87N) was shown to bind calcium with low affinity (K-_{ca}2×10⁵M⁻¹) (Permyakov S. E., et al., (2001) Proteins Eng 14, 785-789).

Such a mutant forms a further preferred embodiment of the invention.

Thus particularly preferred variants for use in the complexes of the invention are D87A and D87N variants of α-lactalbumin, or fragments which include this mutation.

The region which forms the interface between the alpha and beta domains is, in human α-lactalbumin, defined by amino acids 34-38 and 82-86 in the structure. Thus suitable fragments will include these regions, and preferably the entire region from amino acid 34-86 of the native protein.

This region of the molecule differs between the bovine and the human proteins, in that one of the three basic amino acids (R70) is changed to S70 in bovine α-lactalbumin thus eliminating one co-ordinating side chain. It may be preferable therefore, that where the bovine α-lactalbumin is used in the complex of the invention, an S70R mutant is used.

It appears that three molecular events are required to form HAMLET from α-lactalbumin. First, the tightly bound Ca²⁺-ion is released. The apo-protein is then allowed to bind the lipid cofactor, for example, on an ion exchange matrix. Third, the active complex is eluted at high salt and dialysed. The elutes were characterised after repeating this procedure with 14 closely related fatty acids as shown hereinafter only the C18:1:9cis and C18:1:11cis complexes were found to cause apoptosis, and they alone gave distinct novel signals by NMR, indicating that they formed a novel molecular complex. Several other fatty acids were capable of retaining the protein on ion exchange matrices and to stabilize the protein in a partially unfolded conformation, but they did not form biologically active complexes and gave sharper NMR signals as expected from a mixture of protein and fatty acid.

It appears that the unsaturated C18:1cis fatty acids have unique structural features, allowing them to form HAMLET from apo-α-lactalbumin, and suggest that they differ from the other fatty acids in that they offer the correct stereo-specific match. The lack of significant HAMLET formation with a number of closely related fatty acids suggests a highly selective and specific process. Consequently, any other fatty acids used in the complex of the invention should have essentially similar stereospecificity to these unsaturated C18:1cis fatty acids.

The Ca²⁺ binding site is 100% conserved in α-lactalbumin from different species (Acharya K. R., et al., (1991) J Mol Biol 221, 571-581), illustrating the importance of this function for the protein. It is co-ordinated by five different amino acids and two water molecules. The side chain carboxylate of D87 together with D88 initially dock the calcium ion into the cation-binding region, and form internal hydrogen bonds that stabilise the structure (Anderson P. J., et al., (1997) Biochemistry 36, 11648-11654). A loss of either D87 or D88 has been shown to impair Ca2+ binding, and to render the molecule stable in the partially unfolded state (Anderson P. J., et al., (1997) Biochemistry 36, 11648-11654).

Further, as reported hereinafter, mutant proteins with two different point mutations in the calcium-binding site of bovine c-lactalbumin were used. Substitution of the aspartic acid at position 87 by an alanine (D87A) totally abolished calcium binding and disrupted the tertiary structure. After substitution of the aspartic acid by asparagine, the protein (D87N) still bound calcium but with lower affinity and showed a loss of tertiary structure, although not as pronounced as for the D87A mutant (Permyakov S. E., et al., (2001) Proteins Eng 14, 785-789). The mutant protein showed a minimal change in packing volume as both amino acids have the same average volume of 125A³, and the carboxylate side chain of asparagines allow the protein to co-ordinate calcium, but less efficiently (Permyakov S. E., et al., (2001) Proteins Eng 14, 785-789). Both mutant proteins were stable in the apo-conformation at physiologic temperatures but despite this conformational change they were biologically inactive in the apoptosis assay. The results demonstrate that a conformational change to the apo-conformation alone is not sufficient to induce apoptosis.

The structure of α-lactalbumin is known in the art, and the precise amino acid numbering of the residues referred to herein can be identified by reference to the structures shown for example in Anderson et al. supra. and Permyakov et al supra.

Native bovine α-lactalbumin and the Ca²⁺ mutants could be converted to the HAMLET like complex named BAMLET, showing that the same fatty acid stabilised bovine α-lactalbumin in the BAMLET conformation. The conversion yield was lower, however, suggesting that lipid binding to the bovine protein was less efficient. The structural basis for this effect is not clear. Bovine and human α-lactalbumin show 76% amino acid sequence identity and have similar native conformations (Wijesinha-Bettoni R., et al., (2001) J Mol Biol 312, 261-273). The lower conversion yield for BAMLET suggested that the sequence differences influenced the fit between the fatty acid and the bovine protein. The divergent sequences are mainly located in the α-helical region (A-helix 57%, B-helix 50%, C-helix 23% and 3₁₀-helix 25% difference) but this region is unlikely to be involved in fatty acid binding (see Example 3 below).

The applicants believe that the lipid binding site in human α-lactalbumin may be located in the groove between the α-helical and β-sheet domains, which becomes exposed in the apo-protein. This region of the molecule differs between the bovine and the human proteins, in that one of the three basic amino acids (R70) is changed to S70 in bovine α-lactalbumin, thus eliminating one potential coordinating side chain.

The results illustrated herein demonstrate that the change in biologic function requires not just a conformational change of the protein, but also the lipid cofactor. The dual requirements for a change in protein conformation and a lipid cofactor may be important to achieve tissue specificity. The active complex should only be formed in local environments that favour the altered protein fold, and where lipid cofactors are available. In the case of HAMLET, such conditions are present in the stomach of the breast-fed child. The low pH precipitates casein with α-lactalbumin in the apo-conformation, and activates pH sensitive lipases that release oleic acid from the milk phospholipids. It is interesting to note that α-lactalbumin and oleic acid respectively, are the most abundant proteins and fatty acid in human milk. The lipids thus appear to function as “post-secretion chaperones”, involved in the adaptation of proteins to shifting external environment. The need for both a folding change and a tissue specific lipid makes sense in order to protect tissues from the occasional protein folding variant on the loose, and to target the site where the novel function is needed.

As part of the present study, the applicants tested the calcium affinity of the complexes. The obtained calcium affinity of α-lactalbumin was higher than previously reported (about 2 mm as compared to 10 mM and up), but the value found (0.15 M NaCl) falls in the range reported by others at 0.1 M NaCl (Table 1 hereinafter). The difference may be explained by the lower ionic strength as we observed that the Ca²⁺ affinity of α-lactalbumin decreased by a factor of 200 between low and physiological ionic strength. This is expected from salt screening of electrostatic interactions, and similar decreases have been obtained with other negatively charged proteins.

The decrease in Kₐ of α-lactalbumin at physiological salt may also be due to competition between Na⁺ and Ca²⁺ for the same site, as Na⁺ has been reported to bind to and stabilize α-lactalbumin in vitro (Kₐ=100 M⁻¹ at 20° C.,). The lack of near-UV CD signals for D87A in the presence of NaCl was a striking contrast to wild-type α-lactalbumin, suggesting that Na⁺ binds to the Ca²⁺ site of the wild-type protein, and that the D87 side-chain is important also for coordination of this monovalent ion.

Complexes of the invention, as well as HAMLET, retained a high affinity for Ca²⁺ at both low and physiological salt concentrations, showing that they can bind Ca²⁺ without loss of activity. This may appear surprising, as partially unfolded conformations of α-lactalbumin usually are associated with the Ca²⁺-free state. Two possible explanations may be offered. In the first and most likely scenario, α-lactalbumin converts to HAMLET by unfolding and binding of oleic acid with little disturbance of the α-helical domain. The Ca²⁺-binding site may then retain a similar affinity as in the absence of oleic acid. A second possibility is that the Ca²⁺ site is disrupted and that the observed Ca²⁺ binding is explained by the generation of a new Ca²⁺ site in HAMLET. The head group of oleic acid might potentially coordinate calcium together with amino acid residues. In this case, the classical Ca²⁺-binding site in α-lactalbumin would not be needed for Ca²⁺-binding to HAMLET. We find this a less likely explanation, as D87A-BAMLET has the oleic acid bound to the unfolded protein, and thus should form the new Ca²⁺ site, but does not bind Ca²⁺. It appears therefore that the Ca²⁺-binding site is not involved in the conversion of α-lactalbumin to an apoptosis-associated conformation, and that the structural changes associated with Ca²⁺ binding to HAMLET do not hinder the biological function.

In HAMLET, α-lactalbumin retains a partially unfolded conformation as well as a high affinity Ca²⁺ binding site. This apparent paradox sheds new light on the molecular characteristics of α-lactalbumin in the complex. The X-ray structure of the native like apo form shows that the alpha and beta regions are largely intact, while the cleft between them is widened (Chrysina et al., J. Biol. Chem, (2000) 275, 37021-9). As discussed above, the applicants believe that the cofactor such as oleic acid binds in the interface between the alpha and the beta domains, and that the bound cofactor acid locks this region of the molecule, while allowing the α-domain to maintain a native-like conformation. This is supported by the finding illustrated hereinafter that complexes of this type such as HAMLET binds Ca²⁺ while retaining activity against tumor cells. It would appear therefore that HAMLET is therefore in a different molecular state than either the low salt apo α-lactalbumin or the native-like apo form in physiological salt.

Complexes of the invention are useful in a variety of therapeutic applications, including anti-cancer and antibacterial treatments, in particular for treatment of infections of the respiratory tract. For these purposes, the complex is suitably formulated as a pharmaceutical composition and these form a further aspect of the invention.

The complex can be administered in the form of an oral mucosal dosage unit, an injectable composition, or a topical composition. In any case the protein is normally administered together with the commonly known carriers, fillers and/or expedients, which are pharmaceutically acceptable.

In case the protein is administered in the form of a solution or cream for topical use the solution contains an emulsifying agent for the protein complex together with a diluent or cream base. Such formulations can be applied directly to the tumour, or can be inhaled in the form of a mist into the upper respiratory airways.

In oral use the protein is normally administered together with a carrier, which may be a solid, semi-solid or liquid diluent or a capsule. Usually the amount of active compound is between 0.1 to 99% by weight of the preparation, preferably between 0.5 to 20% by weight in preparations for injection and between 2 and 50% by weight in preparations for oral administration.

In pharmaceutical preparations containing complex in the form of dosage units for oral administration the compound may be mixed with a solid, pulverulent carrier, as e.g. with lactose, saccharose, sorbitol, mannitol, starch, such as potato starch, corn starch, amylopectin, cellulose derivatives or gelatine, as well as with an antifriction agent, such as magnesium stearate, calcium stearate, polyethylene glycol waxes or the like, and be pressed into tablets. Multiple-unit-dosage granules can be prepared as well. Tablets and granules of the above cores can be coated with concentrated solutions of sugar, etc. The cores can also be coated with polymers which change the dissolution rate in the gastrointestinal tract, such as anionic polymers having a pka of above 5.5. Such polymers are hydroxypropylmethyl cellulose phthalate, cellulose acetate phthalate, and polymers sold under the trade mark Eudragit S100 and L100.

In preparation of gelatine capsules these can be soft or hard. In the former case the active compound is mixed with oil, and the latter case the multiple-unit-dosage granules are filled therein.

Liquid preparations for oral administration can be present in the form of syrups or suspensions, e.g., solutions containing from about 0.2% by weight to about 20% by weight of the active compound disclosed, and glycerol and propylene glycol. If desired, such preparations can contain colouring agents, flavouring agents, saccharine, and carboxymethyl cellulose as a thickening agent.

The daily dose of the active compound varies and is dependant on the type of administrative route, but as a general rule it is 1 to 100 mg/dose of active compound at personal administration, and 2 to 200 mg/dose in topical administration. The number of applications per 24 hours depend of the administration route, but may vary, e.g. in the case of a topical application in the nose from 3 to 8 times per 24 hours, i.e., depending on the flow of phlegm produced by the body treated in therapeutic use.

The invention further provides a method for treating cancer which comprises administering to cancer cells a complex or a composition as described above.

The invention further provides a method for treating bacterial infections which comprises administering to a patient in need thereof, a complex or a composition as described above.

In the description, the following abbreviations have been used.
GC/MS: Gas chromatography/Mass spectrometry,
EDTA: ethylenediamintetra acetic acid, Tris tris(hydroxymethyl)aminomethane, ANS: 8-Anilinonaphtalene-1-sulfonic acid,
CD: circular dichroism,
UV: ultra violet,
NaCl: sodium chloride,
NMR: nuclear magnetic resonance, ppm: parts per million
FITC: fluorescein isothiocyanate,
TLC: thin layer chromatography,
DEAE: diethylaminoethyl,
HCl: hydrochloric acid,
EGTA: ethylene-bis(oxyethyleneitriol)tetraacetic acid.
FPLC; fast protein liquid chromatography;
PBS, phosphate-buffered saline.

The invention will now be particularly described by way of example with reference to the accompanying diagrammatic drawings in which:

FIG. 1 shows simplified fatty acid structures and in particular line drawings of the unsaturated fatty acids, which were investigated for their ability to produce a HAMLET like molecular complex. C16:1:9cis=Palmitoleic acid, C18:1:6cis=Petroselinic acid, C18:1:9cis=Oleic acid, C18:1:11cis=vaccine acid, C20:1:11cis=Eicosenic acid, C18:1:9trans=Elaidic acid, C18:1:11trans=Trans vaccenic acid, C20:4,5,8,11,15cis=Arachidonic acid, C18:3:6,9,12cis=Gamma linolenic acid, C18:3:9,12,15cis=Linolenic acid, C18:2:9,12cis=Linolenic acid.

FIG. 2 is a series of graphs showing the retention of apo-α-lactalbumin on ion exchange matrices conditioned with individual fatty acids.

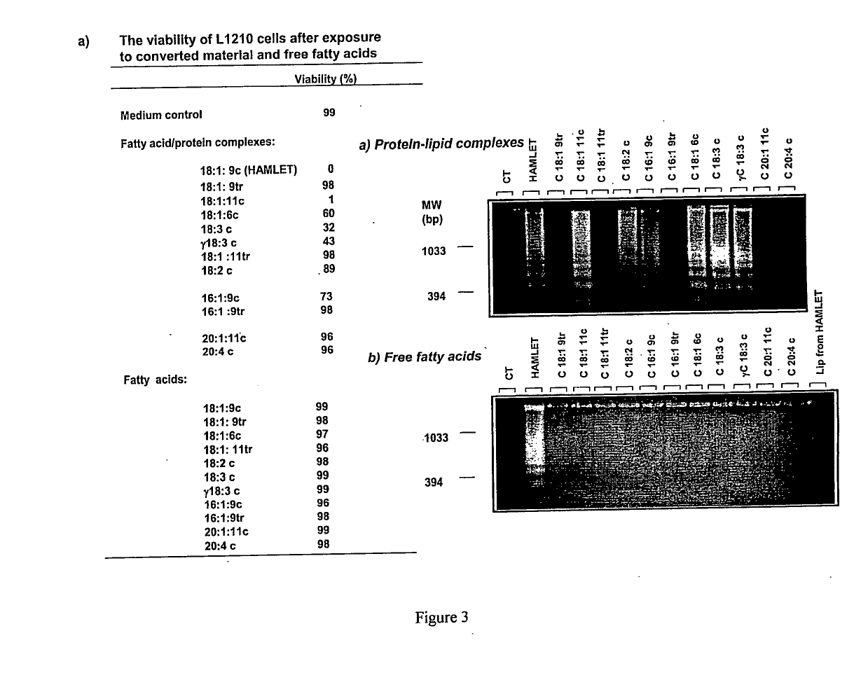

FIG. 3 illustrates tumour cell apoptosis induced by the lipid-protein complexes.

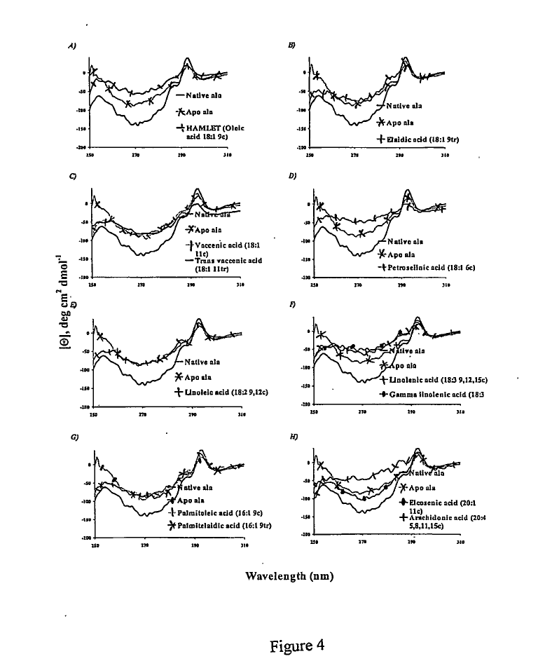

FIG. 4 shows the results of CD spectroscopy to determine the tertiary structure of the fatty acid-protein complexes.

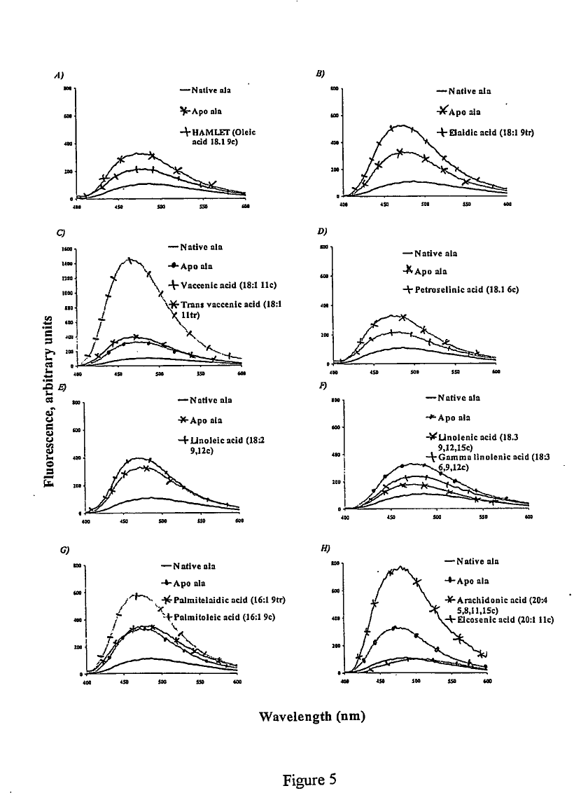

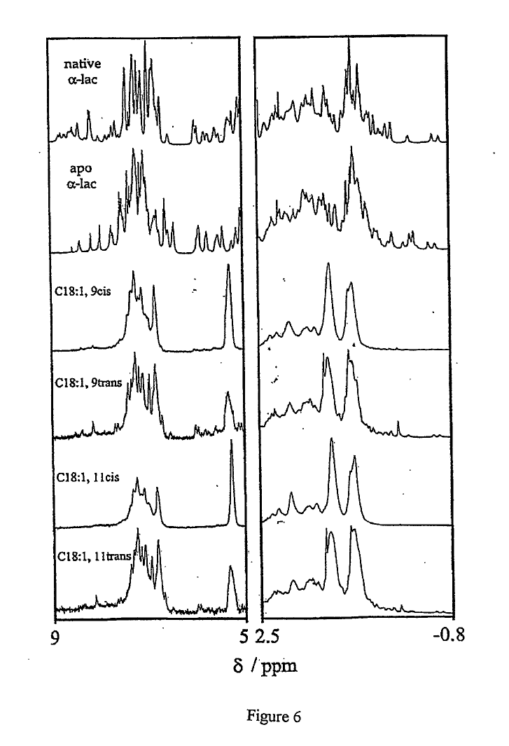

FIG. 5 shows the results of probing of the fatty acid-protein complexes by ANS spectroscopy as an indicator of hydrophobicity.

FIG. 6 shows the results of NMR analysis of complexes.

FIG. 7 illustrates the characterisation of the D87A and D87N mutants of α-lactalbumin, in which panel A shows the structure of the calcium-binding site.

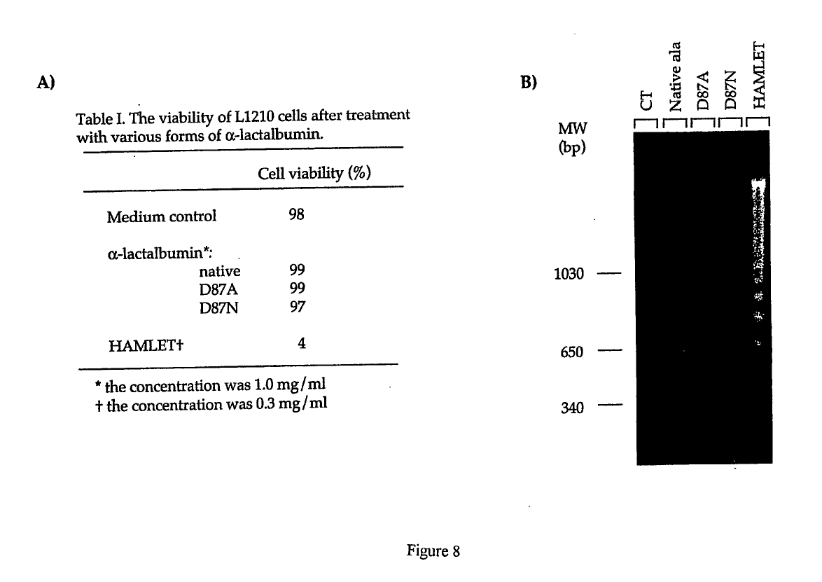

FIG. 8 illustrates biological tests carried out using mutated proteins alone, and shows that they do not induce apoptosis.

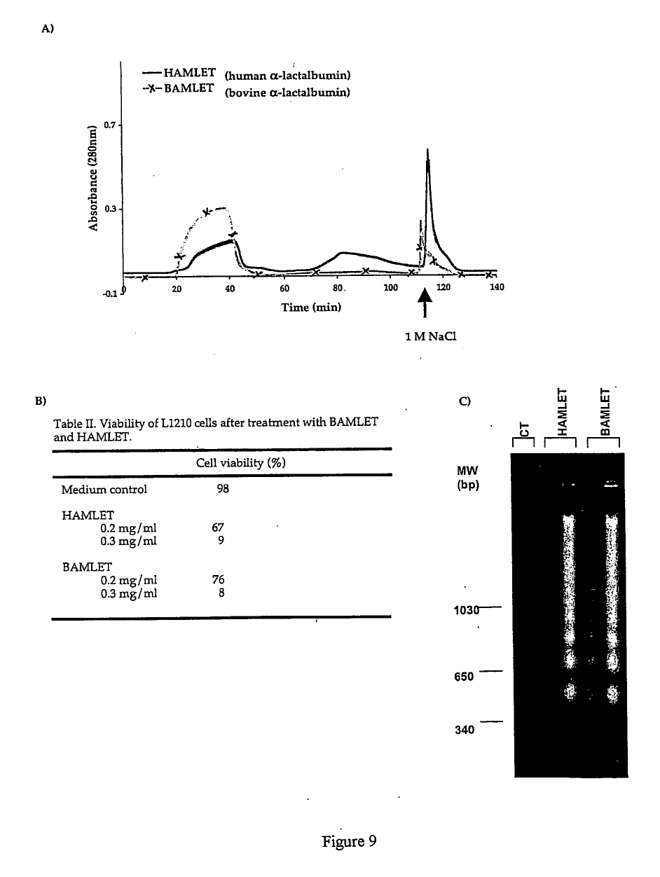

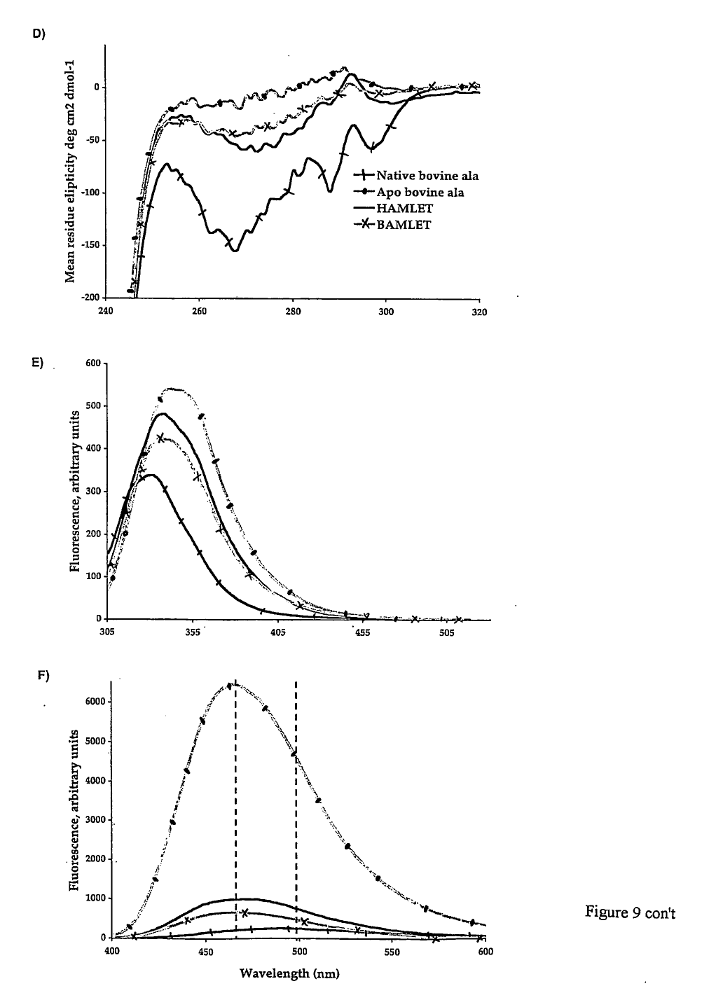

FIG. 9 illustrates the conversion of bovine α-lactalbumin to BAMLET, where panel A shows elution peaks obtained during the preparation, panels B and C relate to the biological testing of BAMLET, panel D shows the results of near UV CD spectroscopy, panel E shows the results of intrinsic fluorescence spectrometry, and panel F shows the ANS spectra of HAMLET and BAMLET.

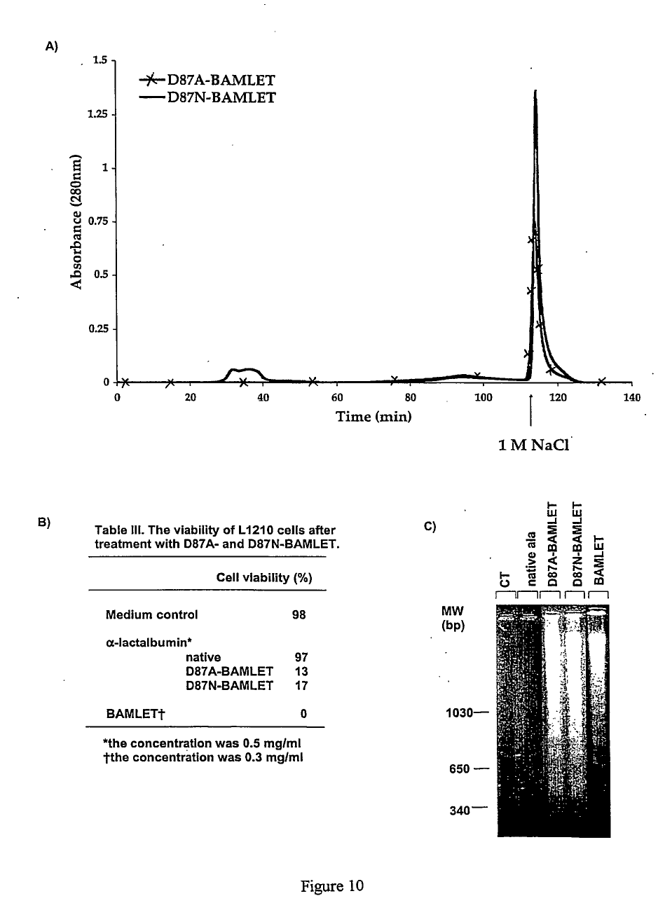

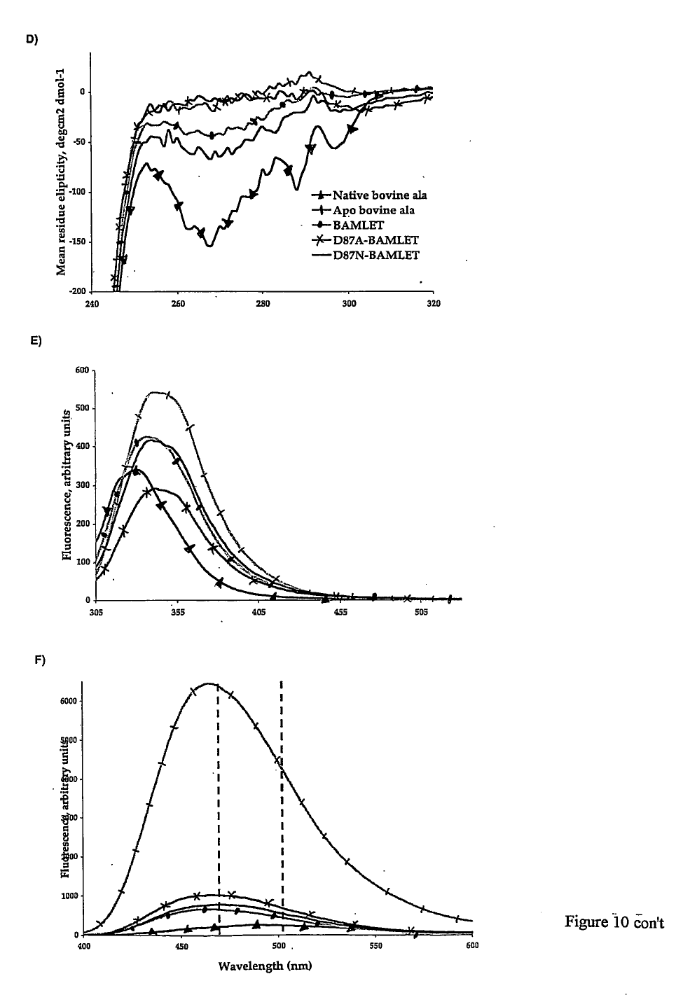

FIG. 10 illustrates the production and test results for D87A and D87N to D87A-and D87N-BAMLET, where panel A shows elution peaks obtained during the preparation, panels B and C relate to the biological testing of these complexes, panel D shows the results of near UV CD spectroscopy, panel E shows the results of intrinsic fluorescence spectrometry, and panel F shows the ANS spectra.

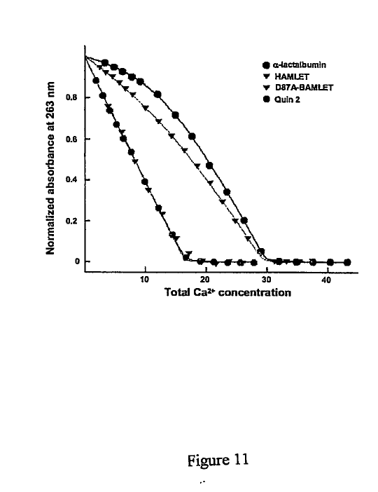

FIG. 11 shows the results of Ca²⁺ titrations in the presence of quin 2. The absorbance is shown as a function of total Ca²⁺ concentration for quin 2 mixed with human α-lactalbumin, HAMLET, or D87A-BAMLET, and quin 2 alone. The solid lines are the fitted curves. The absorbance is normalized using the fitted values for completely Ca²⁺-free and Ca²⁺-loaded system, respectively.

### EXAMPLE 1

Structural variants of oleic acid, and other fatty acids differing in the degree of saturation, carbon chain length and cis/trans conformation (FIG. 1) were compared for their ability to form HAMLET-like complexes from apo-α-lactalbumin.

Apo α-lactalbumin was applied to column matrices that had been pre-conditioned with each indicated fatty acid indicated in FIG. 1 using the method described by M. Svensson, et al., (2000) Proc Natl Acad Sci USA, 97, 4221-6, and the eluate after 1M NaCl was collected. Columns conditioned with C18:1:9cis were used as a positive control (a). All unsaturated fatty acids in the cis conformation retained apo-α-lactalbumin on the column, but with varying efficiency. Unsaturated fatty acids in the trans conformation (C18:1, 9trans, C16:1, 9trans) or saturated fatty acids (C6:0 and C18:0) failed to retain apo-α-lactalbumin on the column.

Results of ion exchange chromatography on fatty acid conditioned matrices are shown in FIG. 2. Unsaturated C18 fatty acids in the cis conformation formed complexes with apo-α-lactalbumin. The C18:1:9cis fatty acid converted >90% of the added apo-α-lactalbumin. The C18:1:11cis fatty acid was somewhat less efficient with a yield of about 70%, and other unsaturated C18 cis fatty acids (C18:1:6cis, C18:2:9,12cis, C18:3:9,12,15cis and gamma C18:3:6,9,12cis gave considerably lower yields.

Trans conformers of C18:1 and saturated fatty acids were practically inactive, however. Only small amounts of protein eluted with high salt from columns conditioned with C18:1:9trans, C18:1:11trans, or the saturated C18:0 fatty acid and they were inactive.

Unsaturated cis fatty acids with shorter (C16:1:9cis) or longer (C20:1:11cis and C20:4:5,8,11,15 cis) carbon chains formed complexes that eluted after 1M NaCl, with yields comparable to C18:1:11cis, but lower than C18:1:9cis. The columns conditioned with the saturated fatty acids C6:0, C14:0 or C16:0 retained no apo-α-lactalbumin.

The results demonstrate that apo-α-lactalbumin interacts in a stereo-specific manner with C18:1 fatty acids, and that C18 fatty acids must be unsaturated and with the double bond in the cis conformation. Unsaturated fatty acids in the trans conformation were inactive, as were the saturated fatty acids. Furthermore the results confirmed that α-lactalbumin in its Ca²⁺-bound form bound only to a very low degree to the C18:1 fatty acid conditioned column. The binding site for the unsaturated cis fatty acid that defines HAMLET is available only in the apo-conformation.

### EXAMPLE 2

Biological Activity

Apoptosis induction was tested using the L1210 leukaemia cell line. Apoptosis induction in L1210 leukaemia cells exposed to the different protein-lipid complexes including the HAMLET control (M. Svensson, et al, (1999) J Biol Chem, 274, 6388-96). HAMLET and the C18:1:11cis complex had killed 99-100% of the cells after six hours (FIG. 3 Table), but the other complexes had little or no effect on the cell viability. Both C18:1cis fatty acid protein complexes induced DNA fragmentation (b), but the C18:1trans fatty acid complexes were inactive. The C16 and C20 unsaturated fatty acid complexes caused an intermediate degree of DNA fragmentation, but no loss of cell viability.

L1210 cells were exposed to lipid extracts derived from HAMLET or from each of the other complexes. No effect on L1210 cell viability (FIG. 3 Table) or DNA fragmentation (c) was detected after six hours exposure to lipid concentrations corresponding to the amount present in 1.0 mg of protein, even though 0.3 mg of HAMLET was sufficient to kill the cells by apoptosis. At very high lipid concentrations, the cells died of necrosis but at no time were there evidence of apoptosis in response to lipids.

Thus the complexes formed with C18:1:9cis and C18:1:11cis fatty acids induced apoptosis more efficiently than the other protein lipid complexes (Table in FIG. 3). Cell viability was reduced from 99% to 0% in six hours at a concentration of 0.3 mg/ml, and DNA fragmentation was observed. Interestingly, other C18:1:cis protein-fatty acid complexes had killed <50% of the cells at this time (Table in FIG. 3). The C18:1:trans fatty acid complexes were inactive in the cellular assay, as were the C20 fatty acid complexes, and the C16 fatty acids complexes showed very low effects on cell viability.

The C18:1:cis fatty acid complexes had induced DNA fragmentation after six hours, suggesting that the cells were dying by apoptosis (FIG. 3a). We were surprised to find evidence of DNA fragmentation also in some cells exposed to the other fatty acid complexes, even though these cells remained viable at six hours.

These results demonstrate that the lipids do not trigger apoptosis, and that HAMLET is defined by both the protein and the lipid. They further demonstrate that only C18:1:9cis and C18:1:11cis, fulfil the criteria for a cofactor in the formation of HAMLET, even though some of the other fatty acids appeared to interact with apo-α-lactalbumin on the ion-exchange matrix.

### EXAMPLE 3

Structural Correlates of the Biologic Activity

The ability to stabilise the protein in an apo-like conformation was determined by CD and ANS spectroscopy, and the structural integration was examined by NMR spectroscopy.

Conformation Assessed by CD Spectroscopy

The complexes eluting after 1M NaCl were examined by near UV CD spectroscopy (M. Svensson, et al., (2000) Proc Natl Acad Sci USA, 97, 4221-6), using native or apo-α-lactalbumin and HAMLET as controls. The native α-lactalbumin control showed the characteristics of a well folded protein, with a minimum at 270 nm arising from tyrosine residues and a maximum at 294 nm arising from tryptophan residues. The apo-α-lactalbumin control had lost most of the characteristic signals, indicating less restrained tyrosines and tryptophans. The C18:3:9,12,15cis, C18:3:6,9,12cis, C20:4:5,8,11,15cis and C18:1:6cis complexes resembled HAMLET with a loss of signal in the tyrosine and tryptophan regions, while remaining complexes were similar to the apo-α-lactalbumin control.

HAMLET was shown to resemble apo-α-lactalbumin, but seems to retain even less of the tertiary structure (FIG. 4a). The other eluted fatty acid-protein complexes showed two main spectral patterns. The C18:1:6cis, C18:3:9,12,15cis, C18:3,6,9,12cis and C20:4:5,8,11,15cis fatty acid complexes resembled HAMLET, while the C18:1:9trans, C18:1:11:cis or trans, C18:2:9,12cis and C16:1:9cis or trans complexes were identical to apo control (FIG. 4b-h). Unconverted apo-α-lactalbumin that eluted in the void was shown to revert to the native state in the presence of Ca²⁺.

These results indicate that all fatty acids, which retain apo-α-lactalbumin on the column, stabilise the protein in a partially unfolded conformation.

Exposure of Hydrophobic Surfaces, as Probed by ANS Spectroscopy

Apo-α-lactalbumin is known to expose hydrophobic side chains, due to the mobility of the β-sheet. The complexes eluting after 1M NaCl were examined by ANS spectroscopy (M. Svensson, et al., (2000) Proc Natl Acad Sci USA, 97, 4221-6) using native or apo-α-lactalbumin and HAMLET as controls. Native α-lactalbumin did not bind ANS as shown by the flat curve and the low signal at 490 nm, but apo-α-lactalbumin showed significant ANS binding with enhanced intensity and a maximum at 470 nm as expected from the increased hydrophobicity of this fold. All of the α-lactalbumin-fatty acid complexes except C20:1:11cis bound ANS. The C18:3:9,12,15cis, gammaC18:3:6,9,12cis and C18:1:6cis complexes resembled HAMLET, while the other fatty acid complexes showed even higher ANS binding.

The apo control showed the expected ANS binding with enhanced intensity and a maximum at 470 nm, while the native protein failed to bind ANS as shown by the flat curve and the low signal at 490 nm. HAMLET bound ANS with a blue shift of the curve, but the peak was lower than for apo-α-lactalbumin, (FIG. 5a).

The C18:1:6cis, C18:3:9,12,15cis, and C18:3:6,9,12cis fatty acid complexes, bound ANS with similar spectral intensity as HAMLET. Some other complexes (C18:1,9trans, C18:1,11cis and trans, C18:2,9,12cis, C16:1,9 cis and trans, C20:4,5,8,11,15cis) showed more intense ANS fluorescence than the apo-α-lactalbumin control. Finally, the C20:1:11cis complex did not bind any ANS (FIG. 5b-h).

To exclude the direct binding of ANS to the fatty acids in the protein-lipid complexes, mixtures of ANS to the fatty acid were subjected to spectroscopy. No ANS—fatty acid interaction was observed (data not shown).

These results demonstrated that the C18:1:9cis fatty acid complex retains the ability to interact with ANS, but the lower intensity suggested that the fatty acid in HAMLET might modify ANS binding. The negative results for C20:1:11cis complex suggested that the longer fatty acid hindered the interaction of hydrophobic surfaces in the protein with ANS.

¹H-NMR Spectroscopy

¹H-NMR was used to resolve the structural basis for the difference in activity between the C18:1cis and the inactive protein-fatty acid complexes (FIG. 6).

Native α-lactalbumin showed the characteristics of a folded and well-ordered protein with narrow lines and significant shift dispersion, a large number of sharp signals in the aromatic region (around 7 ppm) and several out shifted methyl signals (between 0.7 and −0.6 ppm). The apo protein displayed narrow lines and significant shift dispersion with significant variations relative to the native state in the chemical shifts of a large number of resonances. The aromatic and methylated regions are shown in the left and right panels, respectively (M. Svensson, et al., (2000) Proc Natl Acad Sci USA, 97, 4221-6). HAMLET showed broad lines and lack of out shifted methyl signals suggestive of a partially unfolded state, and significantly different from the native protein. The spectrum obtained with the C18:1,11cis protein complex was virtually identical to HAMLET but the spectra of the trans fatty acid complexes showed more narrow lines and out shifted signals suggesting that the conformation of the C18:1:9cis or C18:1:11cis complexes are unique and that although the trans fatty acids bind to apo-α-lactalbumin, they do not alter the conformation so that HAMLET is formed.

The spectrum of both the C18:1,9cis and C18:1,11cis complexes showed broad lines and little shift dispersion. The lines in the aromatic region were clustered and there were no out shifted methyl signals below 0.7 ppm. The broad signals of the fatty acid suggested that they formed an integral part of the complex.

The trans isomer complexes (C18:1:9trans and C18:1:11trans) differed markedly from the C18:1:9cis or C18:1:11cis complexes. Signals from bound fatty acid were detected, but they were smaller than for the cis complexes. The protein lines were narrow, and out shifted both in the methyl and the aromatic regions. These data suggested that the trans fatty acids bind to apo-α-lactalbumin, but do not alter the conformation so that HAMLET is formed.

These results suggest that specific molecular interactions stabilise apo-α-lactalbumin in the HAMLET conformation, and only the unsaturated C18:1:9cis or C18:1:11cis fatty acids have the stereo specific properties required to achieve this conformational switch.

Apo-α-lactalbumin differs from other known lipid-binding proteins in that it contains both α-helical and β-sheet domains. The intracellular lipid-binding protein family have an all β-barrel structure, forming a cavity which binds in a range of fatty acids varying in chain length and saturation (J. Thompson, et al., (1997) J Biol Chem, 272, 7140-7150). Typically, the carboxylate head group of the fatty acids interacts with two to four positively charged amino acids, usually arginines, and the carbon chain is co-ordinated by six to ten hydrophobic amino acids. The crystal structure of human serum albumin has revealed six asymmetrically distributed, fatty acid binding sites within the repeating α-helical domain structure of the protein (S. Curry, et al., (1998) Nature Struc Biol, 5, 827-835). Each hydrophobic pocket is capped at one end by basic or polar side chains, co-ordinating the fatty acid head group. While the binding of fatty acids to human serum albumin causes conformational changes with rotations of the three domains of the protein, and adjustments of side chains to make way for incoming fatty acid (S. Curry, et al., (1998) Nature Struc Biol, 5, 827-835), the molecule does not unfold or change function. We may therefore conclude that the lipid cofactor function in the conversion of α-lactalbumin HAMLET differs both structurally and functionally from these previously known protein lipid interactions.

Tentative fatty acid binding sites were identified based on the three-dimensional structures of native apo-α-lactalbumin. The native α-lactalbumin molecule is a hydrophilic, acidic protein, exposing mainly charged and polar amino acids. Two hydrophobic regions are located in the interior of the globular structure.

One is formed by residues from the C and D helices and the β-sheet domain in the interface between the two domains. The second is formed by residues in the A, B and 30₁₀-helices of the α-domain (FIG. 9) (L. C. Wu, et al., (1998) J Mol Biol, 280, 175-82; M. Saito, (1999) Protein engineering 12, 1097-1104). The crystal and NMR structures of bovine apo α-lactalbumin have revealed a significant structural change in the cleft between the two domains (E. Chrysina, et al., (2000) J Biol Chem, 275, 37021-37029; R. Bettoni-Wijesinha, et al., (2001) J Mol Biol, 307, 885-898) upon Ca²⁺ release. The expansion of the Ca²⁺ binding loop tilts the 3₁₀ helix towards the C helix, resulting in a disruption of the aromatic cluster in the interface between the two domains (Trp 60 and 104, Phe 53 and Tyr 103) (E. Chrysina, et al., (2000) J Biol Chem, 275, 37021-37029). The α-domain, in contrast, remains structured in both the native and the apo-conformations, with near native side chain packing. It seems likely therefore that the C18:1 fatty acid binds in the interface between the α and β domains, and thus stabilises a molten-globule like conformation.

The shape of the hydrophobic pocket suggested that it should favour interactions with bent molecules (FIG. 1). This may indeed explain the inability of the C18:1 trans conformers to form HAMLET. While fatty acids in this cis conformation are u-shaped around the double bond, with both carbon chains projecting in one direction, trans fatty acids are rod shaped around the double bond due to the carbon chains on opposite sides of the double bond. The saturated fatty acids are most flexible with no structural constraints due to the lack of double bonds. The results thus indicate that only the cis conformation allows fatty acids a close stereo-specific fit, and that the additional critical feature of the fatty acid is the carbon chain length. In addition, the pocket is capped by basic residues, which may co-ordinate the polar head groups of the fatty acids, thus orienting the lipid. This interaction is, however not sufficient for activation as the trans and saturated fatty acids, which possess the same charged head group failed to form the active complex. It is highly likely that the stereo specific fit involves both hydrophobic interactions with the lipid tail and electrostatic interactions of the negatively charged head group with basic side chains. Based on the analogy with other fatty acid binding proteins, the fatty acid may bind to HAMLET by electrostatic interactions between its negatively charged head group and basic side-chains in the protein, as well as by van der Waal's contacts and hydrophobic effects with the tail that are optimized with the preferred stereo specific match (C18:1:9cis).

### EXAMPLE 4

Analysis of Variants of α-lactalbumin

The apo-conformation of α-lactalbumin is unstable and the protein reverts to the native state at neutral pH and at the Ca²⁺ concentrations present in the apoptosis assay. In the HAMLET complex, the protein maintains an apo-like conformation, however. As the lipid alone does not trigger apoptosis, it might act simply by stabilising the apo-conformation. A conformational change of the protein might then be sufficient to induce apoptosisr but the unstable nature of the apo conformation has precluded experiments testing the activity of the protein per se.

This question was addressed by site directed mutagenesis of the Ca²⁺ binding site. The bound Ca²⁺ ion is co-ordinated by a constellation of seven oxygen groups that form a distorted pentagonal bipyramid, but a mutation of the Aspartic acid residue at position 87 is sufficient to fully or partially inactivate the Ca²⁺ binding site, generating mutant proteins locked in the apo-conformation (Anderson P. J., et al., (1997) Biochemistry 36, 11648-11654; Veprintsev D. B., et al., (1999) Proteins 37, 65-72; Permyakov S. E., et al., (2001) Proteins Eng 14, 785-789). This study examined if apoptosis can be triggered solely by a conformational change from the native to the apo-state. Furthermore, the importance of the Ca²⁺ binding site for the conversion to HAMLET was investigated.

Native human α-lactalbumin was purified from human milk by ammonium sulphate precipitation and phenyl sepharose chromatography as described (Svensson M., et al., (2000) Proc Natl Acad Sci USA 97, 4221-4226). Apo α-lactalbumin was generated from 25 mg of native α-lactalbumin dissolved at 1.8 mM in Tris¹ (10M Tris/HCl pH 8.5) by addition of 5 mM EDTA to remove bound Ca²⁺. The conformational change was confirmed by near UV CD and ANS spectroscopy. Bovine α-lactalbumin was purchased from Sigma, St. Louis, Mo., USA and used without further purification.

In addition, a mutated bovine protein (D87A and D87N) was expressed in E. coli, purified, folded and lyophilised as described (Anderson P. J., et al., (1997) Biochemistry 36, 11648-11654; Permyakov S. E., et al., (2001) Proteins Eng 14, 785-789).

A column (14 cm×1.6 cm) packed with DEAE-Trisacryl M (BioSepra, France) was attached to a Bio-Logic chromatography system (Bio-Rad Laboratories, Hercules, Calif.), and eluted with a NaCl gradient (buffer A: 10 mM Tris/HCl pH 8.5; buffer B: buffer A containing 1 M NaCl). The matrix was conditioned with oleic acid (Larodan biochemicals, Malmo, Sweden). Ten milligrams was dissolved in 500 μl 99.5% ethanol by sonication (3 minutes using a Branson 2200 bath sonicator, Branson, Danbury, USA). After addition of 10 ml of 10 mM Tris/HCl, pH 8.5, the lipid solution was applied to a newly packed DEAE-Trisacryl M matrix and dispersed through out the matrix using a NaCl gradient.

Ten mg of each of human and recombinant and native bovine α-lactalbumin was dissolved in 10 ml of 10 mM Tris/HCl pH 8.5 and added to the column. The protein fraction eluting after high salt was desalted by dialysis (Spectra/Pore, Spectrum Medical Industries, Laguna Hills, Calif., membrane cut off 3.5 kDa) against distilled water with at least four changes of water in 100-fold volume excess, and then lyophilised.

The products were then subjected to spectroscopic analysis. The proteins or protein fractions were dialyzed against doubly distilled water and lyophilised. Stock solutions were prepared by dissolving the lyophilised material in 10 mM potassium phosphate buffer at pH 7.5, and concentrations determined as the absorbance at 280 nm (A/l=C (mg/ml) where 1 mg/ml of α-lactalbumin is 70 μM). The spectra were recorded at the appropriate dilution.

Circular Dichroism (CD) spectra were obtained on a JASCO J-720 spectro-polarimeter with a JASCO PTC-343 Peltier type thermostated cell holder. Quartz cuvettes were used with 1 cm path length and spectra were recorded at 25° C. between 240 and 320 nm. The wavelength step was 1 nm, the response time δ s and the scan rate was 10 nm per minute. Six scans were recorded and averaged for each spectrum. Baseline spectra were recorded with pure buffer in the cuvette and substracted from the protein spectra.

The mean residue ellipticity qₘ (mdeg×cm²×dmol⁻¹) was calculated from the recorded ellipticity, q, as
qₙ=q/(c·n·1)
where c is the protein concentration in M, n the number of residues in the protein (123 in this case), 1 the path length in nm and q is the ellipticy in degrees.

Fluorescence spectra were recorded at 25° C. on a Perkin Elmer LS-50B spectrometer using a quartz cuvette with 1 cm excitation path length. Intrinsic (tryptophan) fluorescence emission spectra were recorded between 305 and 530 nm (step 1 nm) with excitation at 295 nm. The excitation bandwidth was 3 nm and the emission was 5 nm. ANS fluorescence emission spectra were recorded at 25° C. on a Perkin Elmer LS-50B spectrometer using a quartz cuvette with 1 cm excitation path length, between 400 and 600 nm (step 1 nm) with excitation at 385 nm. Both the excitation and emission bandpass were set to 5 nm. ANS ammonium salt (Fluka, Buchs, Switzerland) was added stepwise and the spectra at 1.5 molar equivalents are shown.

The results are illustrated in FIG. 7. Panel A shows ribbon diagrams of the calcium-binding loop with the co-ordinating side chains shown as darkly shaded lines. In the wild-type protein calcium is co-ordinated by K79, D82,84,87 (arrow) and D88. If D87 is changed to A (arrow), the protein looses its ability to bind calcium. If D87 is changed to N (arrow) the protein can still bind calcium but with low affinity.

The results of the investigation into the tertiary structure of the two mutants is shown in panel B. Spectra were recorded in sodium phosphate buffer without EDTA. Native bovine α-lactalbumin had a minimum at 270 nm arising from tyrosine residues and a maximum at 294 nm arising from tryptophan residues. Apo α-lactalbumin showed the characteristic loss of signal, indicating less restrained tyrosines and tryptophans. The D87A mutant showed an almost complete loss of ellipticity consistent with a partially unfolded conformation. The spectrum of the D87N mutant showed decreased ellipticity in the tyrosine and tryptophan regions, although not to the same extent as the D87A mutant.

The result of intrinsic fluorescence spectroscopy is shown in panel C. Native bovine α-lactalbumin showed an intensity maximum at 335 nm and a shoulder at 320 nm, indicative of tryptophan residues in a folded hydrophobic core, but shifted to 350 in apo α-lactalbumin indicating that the tryptophans are more accessible to the solvent. Both the D87A and the D87N mutant showed intensity maxima at 350 nm resembling apo-α-lactalbumin.

Fluorescence spectra at 1.5 equivalents of ANS are shown in FIG. 7 panel D. Native bovine α-lactalbumin did not bind ANS but resembled ANS added to pure buffer. Apo α-lactalbumin bound ANS with a maximum at 475 nm and significantly enhanced intensity. The D87N and D87A mutants bound ANS with intensity maxima at 475 nm, strongly resembling the spectrum of the apo control.

Thus in summary, the near UV CD spectroscopy, a nearly complete loss of ellipticity was observed demonstrating loss of tertiary structure (FIG. 7b) and the intensity maximum in the intrinsic tryptophan spectrum was shifted towards higher wavelengths (FIG. 7c). The D87A mutant bound ANS as shown by the height of the curve and the shift of the intensity maximum to shorter wavelengths (FIG. 7d). No spectral changes were observed following the addition of excess calcium (1 mM) showing that the D87A mutant did not bind calcium under these conditions (Anderson P. J., et al., (1997) Biochemistry 36, 11648-11654).

The near UV CD spectrum was intermediate between the native and the D87A spectra, but had a significantly reduced ellipticity (FIG. 7b) as compared to native α-lactalbunim, and the intrinsic fluorescence spectrum was red shifted compared to native protein suggesting exposed tryptophans and a loss of tertiary structure (FIG. 7c). The D87N mutant bound ANS demonstrating exposed hydrophobic surfaces (FIG. 7d). Addition of EDTA (1 mM) had only a marginal effect on the near UV CD ellipticity (Permyakov S. E., et al., (2001) Proteins Eng 14, 785-789), showing that the D87N mutant is in a molten globule like state also in the presence of calcium.

It appears that the D87N protein is in an apo-like conformation, but with better defined tertiary structure than D87A.

These results conformed that mutations in the calcium-binding site lock the protein in a molten globule like conformation, which is insensitive to the calcium conditions.

### EXAMPLE 5

Bioassays of Apoptosis

The L1210 (ATCC, CCL 219) cell line was cultured in suspension, as described (Svensson M., et al., (1999) J Biol Chem 274, 6388-6396). The cells were harvested by centrifugation (200 g for 10 min), re-suspended in cell culture medium (RPMI 1640 supplemented with 10% fetal calf serum, non essential amino acids, sodium pyruvate and 50 μg gentamicin/ml, Life Technologies, Gibco BRL, Paisly, United Kingdom) and seeded into 24 well plates (Falcon, Becton Dickinson, N.J., USA) at a density of 2×10⁶/well. The different agonists were dissolved in cell culture medium, without fetal calf serum, and added to the cells (final volume 1 ml per well). Plates were incubated at 37° C. in 5% CO₂ atmosphere and 100 μl of fetal calf serum was added to each well after 30 minutes. Cell culture medium served as a control.

Cell viability was determined by Trypan blue exclusion after six hours of incubation. For analysis, 30 μl of the cell suspension was mixed with 30 μl of a 0.2% trypan blue solution and the number of stained cells (dead cells) per 100 cells was determined by interference contrast microscopy (Ortolux II, Leitz Wetzlar, Germany).

DNA Fragmentation

Oligonucleosome length DNA fragments were detected by agarose gel electrophoresis. The cell suspension remaining after trypan blue (970 μl, 2×10⁶/ml) was lysed in 5 mM Tris, 20 mM EDTA, 0.5% Triton X-100 pH 8.0 at 4° C. for 1 hour and centrifuged at 13,000×g for 15 minutes. DNA was ethanol precipitated over night in −20° C., treated with proteinase K and RNAse, loaded on 1.8% agarose gels and electrophoresed with constant voltage set at 50V over night. DNA fragments were visualised with ethidium bromide using a 305 nm UV-light source and photographed using Polaroid type 55 positive-negative film.

Mutant Proteins

The ability of the mutant proteins to induce apoptosis was tested using the L1210 cell line. The proteins were suspended in cell culture medium at 2 mg/ml and the cell viability was determined after six hours of incubation as was the DNA fragmentation. The HAMLET control induced apoptosis at 0.3 mg/ml but the mutant proteins had no effect (FIG. 8).

FIG. 8, Panel A Table 1 shows the viability of L1210 cells after 6 hours' exposure to the mutant proteins. The mutants were unable to kill the cells even at a concentration of 1.0 mg/ml (c.f the results for BAMLET (see FIG. 9) where the viability reduced from 98% to 4%.

The mutant proteins did not induce DNA fragmentation, but BAMLET stimulated the formation of the characteristic DNA ladder as shown in panel B.

These results demonstrate that the protein without associated oleic acid is not sufficient to induce apoptosis in tumour cells.

### EXAMPLE 6

Preparation of BAMLET (Bovine α-Lactalbumin Made Lethal to Tumour Cells)

In view of the structural homology between the human and bovine proteins it should be possible to convert bovine α-lactalbumin to HAMLET like molecule, with apoptosis inducing properties. Hence, bovine α-lactalbumin was subjected to the conversion conditions previously used for human α-lactalbumin. Bovine α-lactalbumin was treated with EDTA to remove Ca²⁺, subjected to ion exchange chromatography on a C18:1 conditioned column and eluted with a NaCl gradient. A large portion of the applied material eluted in the void but about 40% formed a sharp peak after 1M NaCl (arrow FIG. 9a). The eluate after high salt was saved for analysis. Human α-lactalbumin was converted with higher efficiency.

A large proportion of the applied material eluted in the void (about 60%), but a small sharp peak eluted after 1M NaCl (FIG. 9a).

The apoptosis-inducing activity of the high salt peak, named BAMLET, was investigated using the L1210 mouse leukemia cell line as described above. Loss of cell viability and DNA fragmentation were used as end points. The L1210 cells died rapidly when exposed to HAMLET (0.3 mg/ml) and DNA fragmentation was induced. The L1210 cells were equally sensitive to BAMLET.

BAMLET reduced cell viability from 99% to 12% at 0.3 mg/ml, after six hours incubation and induces DNA fragmentation (FIG. 9b). There was no apparent difference in efficiency of apoptosis induction between HAMLET and the bovine equivalent (FIG. 9b).

The tertiary structure of BAMLET was assessed using near UV CD spectroscopy. Native bovine α-lactalbumin shared the characteristic spectrum of a well-folded protein with tyrosine dip and tryptophan peak, and native bovine α-lactalbumin did not bind ANS. The bovine apo protein had a reduced signal in both the tyrosine and tryptophan regions, indicative of a partially folded protein with flexible side chains, and significant ANS binding with the maximum at 470 nm and enhanced intensity. The bovine complex strongly resembled both HAMLET and the apo-control (FIG. 9c). The native and apo controls were as in FIG. 7. HAMLET showed decreased ellipticity in the tyrosine and tryptophan regions characteristics of a partially unfolded protein. BAMLET had spectra similar to the apo control and to HAMLET, indicating flexible aromates.

The intrinsic fluorescence spectrum native bovine α-lactalbumin had an intensity maximum at 320 nm, as expected from tyrptophan residues in the folded protein. The apo-protein had an intensity-maximum at 345 nm and a shoulder at 360 nm, indicating that the tyrptophans are more exposed (FIG. 9e). HAMLET showed an intrinsic fluorescence intensity maximum at 345 nm and a shoulder at 360 nm indicating solvent exposed tryptophans. The spectrum of BAMLET was similar to that of HAMLET but without the shoulder. The results indicate that tryptophan residues are shielded from solvent in the native protein, but are more solvent exposed in the apo control, HAMLET and BAMLET.

The ANS spectrum of HAMLET was blue-shifted with the intensity maximum at 475 nm and increased quantum yield, indicative of ANS binding. The spectrum of BAMLET was virtually identical to that of HAMLET. The results indicate exposed hydrophobic surfaces in the apo control, HAMLET and BAMLET but not in the native protein. The bovine complex and HAMLET had virtually identical spectra and bound ANS (FIG. 9f) resembling the apo control.

It can be concluded that bovine apo-lactalbumin can be converted in the presence of C18:1 to a molecular complex that induces apoptosis, and named this complex BAMLET (Bovine α-lactalbumin made lethal to tumour cells).

### EXAMPLE 7

Conversion of the D87A and D87N Mutants to BAMLET

The D87A mutant described-above was applied to a C18:1 conditioned ion exchange column without EDTA and the column, and most of the applied protein eluted as a sharp peak after high salt (arrow) (FIG. 10a). The D87N mutant was first treated with EDTA to remove residual calcium and applied to the column. A small portion of applied protein eluted in the void volume, but the majority eluted as a sharp peak after high salt (arrow). The eluted protein-lipid complexes amounting to >90% of applied D87A and >95% of the D87N protein were named D87A- and D87N-BAMLET.

These complexes were testing in the apoptosis and DNA fragmentation assays described above. Table III (FIG. 10b) shows the loss of viability after 6 hours' exposure of L1210 cells to d87A- and D87N-BAMLET. At 0.5 mg/ml D87A- and D87N-BAMLET reduced the viability from 98% and 13% and 17% respectively. The D87A-and D87N-BAMLET induced DNA fragmentation similar to the BAMLET control (FIG. 10c). The LD₅₀ies of the mutant complexes (0.4 mg/ml) were slightly higher than for BAMLET and HAMLET (0.2 mg/ml).

Spectroscopic Characterisation of the D87A- and D87N-BAMLET

The conformations of D87A and D87N-BAMLET were compared to native and apo-bovine α-lactalbumin and to BAMLET. Near UV CD spectroscopy was carried out on the complexes, with the native, apo and BAMLET controls as in FIGS. 7 and 9. The D87A-BAMLET spectrum was very similar to the unconverted D87A protein with virtually no ellipiticy showing that D87A-BAMLET is in the apo configuration. The spectrum of D87N-BAMLET was virtually identical to that of BAMLET with reduced ellipiticy in both the tyrosine and tryptophan region (FIG. 10d).

Intrinsic tryptophan fluorescence spectroscopy was conducted with the native, apo-α-lactalbumin and BAMLET controls as in FIGS. 1 and 3. The results (FIG. 10e) with D87A- and D87N-BAMLET showed intensity maxima at 345 nm with shoulder at 355 nm strongly resembling BAMLET and the human apo-α-lactalbumin control, suggesting that tryptophans are accessible to solvent.

ANS spectroscopy was conducted, with the native, apo- and BAMLET controls as in FIGS. 7 and 9. Both D87A- and D87N-BAMLET bound ANS, with spectra resembling BAMLET and the apo-α-lactalbumin control. D87A-D87N-BAMLET bound ANS with the intensity maximum shifted to 470 nm and an increased quantum yield compared to the native control, indicating exposed hydrophobic surfaces in all proteins (FIG. 10f).

These results demonstrated that D87A- and D87N-BAMLET maintain the partially folded state with structural and functional properties resembling HAMLET and BAMLET. Calcium removal prior to oleic acid treatment was not required for the D87A mutant because the protein is most likely free from bound calcium and largely rests in the apo form.

As the complexes maintained their biologic activity, it appears that a functional calcium-binding site is not required for the apoptotic function of this complex.

### EXAMPLE 8

Calcium Binding to Alpha-Lactalbumin and the HAMLET Complex

The chromophoric chelator quin 2 was obtained from Fluka Chemie AG, Buchs, Switzerland. Other chemicals were of highest obtainable laboratory quality. To produce Ca²⁺-free buffers, membrane tubing (M.w. cutoff: 3500, Spectrum Medical Industries Inc., LA, Ca, USA, boiled four times in doubly distilled water before use) was filled with 10 ml Chelex 100 (Biorad, Richmond, Calif., USA), sealed, and stored in the solutions to absorb Ca²⁺.

Native human α-lactalbumin was purified from human milk by ammonium sulphate precipitation and phenyl sepharose chromatography as described (Svensson et al., PNAS. 2000, 97, 4221-6). Bovine α-lactalbumin was both purchased from Sigma, St. Louis, Mo., USA and purified from bovine milk using ammonium sulphate precipitation and phenyl speharose chromatography (Svensson et al., supra.). The mutated proteins (D87A and D87N) were expressed in E. coli, purified, folded and lyophilised as described (Anderson et al., 1997), (Permyakov et al., Protein Eng. (2001, 14:785-9). The purity of the protein was assayed by SDS-PAGE and agarose gel electrophoresis, and by NMR spectroscopy.

Apo human or bovine α-lactalbumin for Ca²⁺-binding studies was generated by dissolving α-lactalbumin in doubly distilled water containing a 10-fold molar excess of EGTA at pH 8.0; The sample was applied to a G-25 gel filtration column after an aliquot of saturated NaCl (calcium-depleted) and eluted by doubly distilled water. The sample was passed through the saturated NaCl to reduce binding of EGTA to the protein, and protein free from both Ca²⁺ and EGTA eluted in the water. The residual calcium content was below 0.1 equivalents as estimated from the titration in the presence of quin2, as described below.

Calcium- and EDTA-free HAMLET was generated as described before, with the following adaptions. All buffers were stored with chelex (preparation described above) on a tipping board for a minimum of five days before use. Only plastic vials were used. The FPLC system (Biorad Biologic, Richmond, Calif., USA), including the 20 ml ion exchange column and all tubings, was washed with 2 volumes of 100 mM EDTA, pH 8.5, and then rinsed with at least 10 volumes of millipore water. This was followed by 2 volumes of buffer before the application of oleic acid. EGTA-free apo alpha-lactalbumin (20 mg) in 80 ml calcium-free buffer was applied to the column in 4 consecutive runs (20 ml in each run). The collected fractions were pooled, dialysed, lyophilized and checked for activity on tumor cells as described above. The calcium content was assayed using atomic absorption spectroscopy or titration in the presence of quin 2 (described below). The product was also characterized by agarose electrophoresis and ¹H-NMR.

To study the Ca²⁺ binding equilibrium, the proteins listed in table 1 including the D87A BAMLET obtained as described in Example 7, and HAMLET, was titrated with calcium in the presence of a chromophoric chelator, quin 2, for which the absorbance at 263 nm decreases (approx. 85%) upon Ca²⁺-binding. The method relies on competition for calcium between the protein and chelator, and can be used to quantitate high affinity sites (ca. 10⁷-5·10⁹ M⁻¹ at low salt, and ca. 5 10⁵-1·10⁸ M⁻¹ at 0.15 NaCl) when quin 2 is the chelator. The exact concentration of the chelator solution (in the range of 25-30 μM) was calculated from the absorbance at 239.5 nm in the presence of excess calcium (using ε_{239.5}=4.2×10⁴ L mol⁻¹ cm⁻¹ for quin 2). Lyophilised protein was dissolved in calcium free (<1 μM Ca²⁺) chelator solution at a concentration of 25-30 μM. The absorbance at 263 nm (A₂₆₃) was recorded for the protein/chelator solution using a UV/Vis 920 spectrophotometer (GBC Scientific Equipment Pty Ltd, Victoria, Australia). Calcium solution (3 mM CaCl₂ in 2 mM Tris/HCl, pH 7.5, with or without 0.15 M NaCl) was added in portions of 4 μl. A₂₆₃ was recorded after each calcium addition.

The titration was continued until no absorbance change was seen for the last five additions. A more concentrated Ca²⁺ stock (10 mM) was used at the end of the titrations of the proteins in 0.15 M NaCl. The data was analysed by least squares fitting directly to the measured quantity, absorbance versus total calcium concentration, using the CaLigator software (Andre and Linse, 2002).

The Ca²⁺ affinity of α-lactalbumin has been extensively studied, mainly for the bovine protein. The reported values (Table 1) vary between 2.5×10⁶ and 5.7×10⁸ M⁻¹, probably reflecting the experimental conditions in terms of temperature, buffer, ionic strength, salt, and protein preparation.

The titration curve was very sensitive to the differences in affinity between chelator and protein, so this ratio can be obtained from the data with high precision. Normalized data (absorbance versus total Ca²⁺ concentration) at low salt are shown in FIG. 11, and the resulting Ca²⁺-binding constants for the proteins at physiological (0.15 M NaCl) and low (no added) salt are listed in Table 1. We found a Ka of 1.8·10⁹ M⁻¹ at low salt (K_{D}=0.56·10⁻⁹ M). The Ca²⁺ ion bound more weakly at physiological salt concentrations (0.15 M NaCl): Kₐ=8.3·10⁶ M⁻¹ (K_{D}=1.2·10⁻⁷ M).

HAMLET and BAMLET were shown to bind Ca²⁺ (FIG. 11). The calcium-binding constant for HAMLET was Kₐ=5.9·10⁹ M⁻¹ (K_{D}=1.7·10⁻⁹ M) at low salt and Kₐ=5.3.10⁶ M⁻¹ (K_{D}=1.9·10⁻⁷ M) at physiological salt concentrations. Ca²⁺-titration data for D87A-BAMLET showed no difference compared to the titration of quin 2 alone (FIG. 11), confirming that D87A-BAMLET has lost the functional Ca²⁺ binding site.

It appears that HAMLET maintains a high calcium affinity in both a low and physiological salt environment. Hence, the calcium affinity is only 3 times lower for HAMLET than for α-lactalbumin when no salt is added and 1.6 times lower at physiological salt concentration.

TABLE 1
Calcium association constants (Kₐ) for α-lactalbumin or
HAMLET at different conditions. First five rows: this work,
mean of two experiments.

| Protein | Kₐ (M⁻¹) | Buffer | Salt | pH | T (K) | Method |
|---|---|---|---|---|---|---|
| α-lac | 1.8 · 10⁹ | 2 mM | — | | | chelator |
| human | | Tris/HCl | | | | |
| α-lac | 8.3 · 10⁶ | 2 mM | 150 | | | chelator |
| human | | Tris/HCl | mM | | | |
| | | | NaCl | | | |
| HAMLET | 5.9 · 10⁸ | 2 mM | — | | | chelator |
| | | Tris/HCl | | | | |
| HAMLET | 5.3 · 10⁶ | 2 mM | 150 | | | chelator |
| | | Tris/HCl | mM | | | |
| | | | NaCl | | | |
| D87A- | n.d.* | 2 mM | — | | | chelator |
| BAMLET | | Tris/HCl | | | | |
| α-lac | 4.3 · 10⁸ | 10 mM | — | 7.5 | 298 | ITC |
| bovine | | Tris/HCl | | | | |
| αlac | 5.7 · 10⁸ | 10 mM | — | 7.5 | 298 | ITC |
| goat | | Tris/HCl | | | | |
| α-lac | 2.85 · 10⁸ | 10 mM | — | 8 | — | DSC |
| bovine | | Tris/HCl | | | | |
| α-lac | 4.8 · 10⁷ | 10 mM | | 7.8 | | EGTA, |
| bovine | | Ammoinium | | | | Fluoroscence |
| | | Bicarbonate | | | | |
| α-lac | 2.0 · 10⁷ | H₂O | 100 | 7 | 294 | EDTA, ⁴³Ca-NMR |
| bovine | | | mM | | | |
| | | | KCl | | | |
| α-lac | 2.0 · 10⁷ | H₂O | 100 | 7 | 294 | EDTA, ⁴³Ca-NMR |
| human | | | mM | | | |
| | | | KCl | | | |
| α-lac | 2.7 · 10⁶ | 20 mM Tris | — | 7.5 | 298 | 1) Fluoescence |
| bovine | | | | | | 2) Hummel & |
| | | | | | | Dryer method |
| α-lac | 1) | 5 mM Tris, | — | 7.2 | 298 | CD 270 nm |
| 1) | 2.5 · 10⁸ | 0.1 mM EDTA | | | | |
| bovine | 2) | | | | | |
| 2) | 3 · 10⁸ | | | | | |
| human, | 3) | | | | | |
| 3) goat | 2.8 · 10⁸ | | | | | |
| bovine | 2.0 · 10⁷ | H₂O | 100 | 8 | 294- | EGTA ¹³C-NMR |
| | | | mM | | 298 | |
| | | | KCl | | | |
| bovine | 1) | 50 mM Hepes | — | 8 | 1) | fluorescence |
| | 2.0 · 10⁷ | | | | 310 | |
| | 2) | | | | 2) | |
| | 4.0 · 10⁸ | | | | 293 | |
| bovine | 2.5 · 10⁶ | 20 mM Tris | — | 7.5 | 298 | microcalorimetry |

1. A biologically active complex comprising alpha-lactalbumin or a variant of alpha-lactalbumin (α-lactalbumin) which is in the apo folding state, or a fragment of either of any of these, and a cofactor which stabilises the complex in a biologically active form, provided that any fragment of α-lactalbumin or a variant thereof comprises a region corresponding to the region of α-lactalbumin which forms the interface between the alpha and beta domains, and further provided that when the complex comprises full length α-lactalbumin or a variant of α-lactalbumin in which the calcium binding site has been modified so that the affinity for calcium is reduced, or it is no longer functional, the cofactor is other than C18:1:9 cis fatty acid.

2. A complex according to claim 1 wherein the cofactor is a cis C18:1:9 or C18:1:11 fatty acid or a different fatty acid with a similar configuration.

3. A complex according to claim 1 wherein the cofactor is C18:1:11 fatty acid.

4. A complex according to claim 1 which comprises a fragment of α-lactalbumin or a variant thereof, which fragment includes a region corresponding to the region of α-lactalbumin which forms the interface between the alpha and beta domains.

5. A biologically active complex according to claim 1 which is obtainable by combining
(i) a cis C18:1:9 or C18:1:11 fatty acid or a different fatty acid with a similar configuration; and
(ii) α-lactalbumin from which calcium ions have been removed, or a variant of α-lactalbumin from which calcium ions have been removed or which does not have a functional calcium binding site; or a fragment of either of any of these, provided that any fragment comprises a region corresponding to the region of α-lactalbumin which forms the interface between the alpha and beta domains, and further provided that when (ii) is full length α-lactalbumin or a variant of α-lactalbumin in which the calcium binding site has been modified so that the affinity for calcium is reduced, or it is no longer functional, (i) is other than C18:1:9 cis fatty acid.

6. A complex according to claim 1 which includes a variant of α-lactalbumin in which the calcium binding site has been modified so that the affinity for calcium is reduced, or it is no longer functional, and in which the cofactor is C18:1:11 fatty acid.

7. A complex according to claim 6 wherein the variant has a mutation at a position corresponding to at least one of the K79, D82, D84, D87 or D88 residues.

8. A complex according to claim 7 which includes a D87A or D87N variant of α-lactalbumin.

9. A complex according to claim 1 which comprises a fragment of α-lactalbumin or a variant thereof, and where the fragment includes the entire region from amino acid 34-86 of the native protein.

10. A complex according to claim 1 wherein the α-lactalbumin is human or bovine α-lactalbumin or a variant of either of these.

11. A complex according to claim 10 wherein the α-lactalbumin is human α-lactalbumin.

12. A complex according to claim 11 wherein the α-lactalbumin is mutant bovine α-lactalbumin which includes an S70R mutation.

13. A complex according to claim 1 which further comprises calcium ions.

14. A pharmaceutical composition comprising a complex according to claim 1 in combination with a pharmaceutically acceptable carrier.

15. A method for treating cancer which comprises administering to cancer cells a complex according to claim 1.

16. (canceled)
